(19) United States
(12) Patent Application Publication
(10) Pub. No.: US 2011/0142811 A1
(43) Pub. Date: Jun. 16, 2011

(54) MEASLES VIRUS FOR THE ELIMINATION OF UNWANTED CELL POPULATIONS

(75) Inventors: Guy UNGERECHTS, Heidelberg (DE); Christof Von Kalle, Schallstadt (DE); Sascha Bossow, Heidelberg (DE)

(73) Assignee: DEUTSCHES KREBSFORSCHUNGSZENTRUM, Heidelberg (DE)

(21) Appl. No.: 12/639,536

(22) Filed: Dec. 16, 2009

(51) Int. Cl. C12N 7/01; C12N 15/45; C12N 15/86; C12N 5/00; A61K 35/76; A61K 48/00; A61P 35/04

(52) U.S. Cl. 424/93.21; 435/235.1; 536/23.72; 435/320.1; 435/325; 424/93.2; 514/44 R

(57) ABSTRACT

The present invention concerns recombinant measles virus as a therapeutic, in particular a recombinant measles virus comprising a suicide gene in its genome, a mutated viral hemagglutinin, and a specificity domain specifically recognizing a cancer antigen. Furthermore, it relates to a polynucleotide coding for such a recombinant measles virus and a vector comprising said polynucleotide. The current invention also relates to a method for the manufacture of and a medicament comprising said recombinant measles virus, as well as a method for treating a solid tumor using said recombinant measles virus.

The present invention concerns recombinant measles virus as a therapeutic, in particular a recombinant measles virus comprising a suicide gene in its genome, a mutated viral hemagglutinin, and a specificity domain specifically recognizing a cancer antigen. Furthermore, it relates to a polynucleotide coding for such a recombinant measles virus and a vector comprising said polynucleotide. The current invention also relates to a method for the manufacture of and a medicament comprising said recombinant measles virus, as well as a method for treating a solid tumor using said recombinant measles virus.

Measles virus (MeV) is an enveloped, negative-strand RNA-virus from the family Paramyxoviridae and the causative agent of measles (rubeola). Infections with wild-type MeV have been associated with spontaneous remissions in patients with lymphoma and leukemia. These observations motivated the development of this virus as an experimental cancer therapeutic. In clinical settings, oncolytic effects of MeV have been documented on a wide array of cancer types, and clinical trials testing MeV in the treatment of ovarian cancer, myeloma and glioma are on the way.

In order to improve its clinical properties, several modifications have been introduced into MeV: to decrease generalized infection of body cells with MeV, recombinant viruses expressing “blinded” hemagglutinins, i.e. hemagglutinins that are unable to interact with their cognate receptors CD46/SLAM, have been introduced (Nakamura et al. (2004) Nat Biotech 22(3):331-336. On a second route of development, specificity-modifying domains, altering the binding activity of the viral hemagglutinin and thus conferring cell-type specificity, have been fused to the hemagglutinin (Schneider et al. (2000) J Virol 74, 9928-9936; Hammond et al. (2001) J Virol 74, 2087-2096; Bucheit (2003) Mol Ther 7, 62-72; Peng et al. (2004) Gene Ther 11, 1234-1239; Nakamura et al. (2005) Nat Biotech (23)₂, 209-214.

Furthermore, there is also a need in the art to further increase the lytic potential of recombinant MeV, i.e. to increase the efficacy of MeV in the killing of target cells. What is more, it is desirable to have a possibility to control the spread of a diagnostic and/or therapeutic MeV infection. To satisfy these needs, “aimed” viruses, carrying a suicide gene in their genomes, have been devised (Ungerechts et al. (2007) Mol Ther 15, 1991-1997).

The present invention, now, relates to a recombinant measles virus comprising a suicide gene in the viral genome, a viral hemagglutinin comprising at least one amino acid exchange preventing or reducing the binding activity of the said viral hemagglutinin, and a specificity domain which specifically recognizes a cell surface antigen specific for a solid cancer.

The term “recombinant measles virus” refers to a measles virus comprising in the viral genome at least one suicide gene as well as envelope protein genes which are modified as described herein below. Preferably, the viral envelope shall comprise the envelope protein encoded by the said modified genes and, preferably, said modified envelope proteins are modified viral hemagglutinins. Preferably, said recombinant MeV is derived from a vaccination strain of MeV, and most preferably said recombinant MeV is derived from strain Edmonston B (SEQ ID NO: 6).

The term “suicide gene” refers to a gene, the expression of which in a cell is or can be lethal for that cell. Lethality may be constitutional, i.e. depend solely on the expression of the suicide gene, or conditional, i.e. in addition depend on the presence of a second substance in the cell, e.g. a non-toxic compound that is converted into a toxic drug by the product of the suicide gene. Preferably, the conditional suicide gene is Escherichia coli purine nucleoside phosphorylase (PNP), mutant bacterial cytosine deaminase (bCD), or a beta-Glucuronidase (βG). PNP is a prodrug convertase, meaning that it converts e.g. 6-methylpurine-2′-deoxyriboside (MeP-dR) to 6-methylpurine (MeP)(Hébrard, C. et al. (2009): Development of gene therapy in association with clinically used cytotoxic deoxynucleoside analogues, Cancer Gene Therapy 16, 541-550). MeP is a diffusible substance and is metabolized to toxic adenosine triphosphate analogs, which inhibit DNA, RNA, and/or protein biosynthesis. Cytosine deaminase is an enzyme that can convert 5-fluorocytosine into the chemotherapy agent 5-fluorouracil (5-FU) (Gnant, M. F. X. et al. (1999): Systemic Administration of a Recombinant Vaccinia Virus Expressing the Cytosine Deaminase Gene and Subsequent Treatment with 5-Fluorocytosine Leads to Tumor-specific Gene Expression and Prolongation of Survival in Mice, Cancer Research 59, 3396-3403). 5-FU molecules are able to diffuse across the cell membrane into adjacent cells without passing through the gap junctions to produce a powerful bystander effect. Beta-Glucuronidase converts e.g. glucuronide prodrugs of doxorubicine to free doxorubicine, a chemotherapeutic compound. It is, however, also envisaged by the current invention, that the product of the suicide gene in a cell does not induce cell death but a cessation of multiplication of said cell, e.g. by causing cell cycle arrest.

The term “viral genome” refers to a genome of a MeV comprising at least the six viral measles genes coding for nucleocapsid protein N, phosphoprotein P, matrix protein M, fusion protein F, hemagglutinin H and Large protein L necessary for the production of progeny virus in a suitable host cell comprising said genome (see example 1).

The term “viral hemagglutinin” or “HA” as used herein relates to polypeptides transcribed and translated by a cell from a hemagglutinin-coding polynucleotide comprising a nucleic acid sequence which encodes the MeV H protein (nucleic acid and protein sequence: Genbank Acc No: Z66517.1, GI:1041617) and comprising at least one, at least two, at least three, at least four, at least five amino acid exchange(s) as compared to the MeV H protein. Said at amino acid exchange(s) prevent or reduce the binding activity of said viral hemagglutinin to its natural receptors CD46 or SLAM on the cell. Preferably, said mutation(s) are selected from a list consisting of exchange of the arginine at position 533 for an alanine (R533A), the tyrosine at position 481 for alanine (Y481A), the serine at position 548 for a leucine (S548L), the phenylalanine at position 549 for a leucine (F549L), the proline at position 497 for a serine (P497S), and the tyrosine at position 543 for an alanine (Y543A). The biological function of said viral hemagglutinin is a reduced or absent binding to the cognate receptors on the cell, CD46 and/or SLAM and/or the recently postulated third receptor EpR (Leonard et al. (2008) J Clin Invest 188, 2448-2458), and incorporation of said viral hemagglutinin into the membrane of the MeV. Suitable assays to determine if a protein is incorporated into the membrane of a MeV and if such protein binds to CD46 and/or SLAM are found in Nakamura et al. (2005, Nat Biotech (23)2, 209-214) and Leonard et al. (2008, J Clin Invest 188, 2448-2458).

It is to be understood that a polypeptide having an amino acid sequence as the viral hemagglutinin protein may, due to the degenerated genetic code, also be encoded by other HA-coding polynucleotides as well.

Moreover, the term “viral hemagglutinin” as used in accordance with the present invention further encompasses variants of the aforementioned specific polypeptides encoded by HA-coding polynucleotide variants. Said HA-coding polynucleotide variants may represent orthologs, paralogs or other homologs of the specific HA-coding polynucleotides. The HA-coding polynucleotide variants, preferably, comprise a nucleic acid sequence characterized in that the sequence can be derived from the aforementioned specific nucleic acid sequences by at least one nucleotide substitution, addition and/or deletion, whereby the variant nucleic acid sequence shall still encode a polypeptide having the activities as specified above. Variants also encompass HA-coding polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N.Y. (1989), 6.3.1-6.3.6. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford.

Alternatively, HA-coding polynucleotide variants are obtainable by PCR-based techniques such as mixed oligonucleotide primer-based amplification of DNA, i.e. using degenerated primers against conserved domains of the polypeptides of the present invention. Conserved domains of the polypeptide of the present invention may be identified by a sequence comparison of the nucleic acid sequence of the HA-coding polynucleotide or the amino acid sequence of the polypeptide of the present invention with sequences of other viral glycoproteins, preferably from other members of the virus family Paramyxoviridae. As a template, DNA or cDNA from viruses, bacteria, fungi, plants or animals may be used. Further, variants include HA-coding polynucleotides comprising nucleic acid sequences which are at least 70%, at least 75%, at least 80%, at least 85%, at least 90%, at least 95%, at least 98% or at least 99% identical to the nucleic acid sequences of the aforementioned gene coding for the MeV HA protein (Genbank Acc No: Z66517.1, GI:1041617). Moreover, also encompassed are HA-coding polynucleotides which comprise nucleic acid sequences encoding amino acid sequences which are at least 70%, at least 75%, at least 80%, at least 85%, at least 90%, at least 95%, at least 98% or at least 99% identical to the amino acid sequence of MV HA. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which are part of the GCG software packet [Genetics Computer Group, 575 Science Drive, Madison, Wis., USA 53711 (1991)], are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A HA-coding polynucleotide comprising a fragment of any of the aforementioned nucleic acid sequences is also encompassed as a HA-coding polynucleotide encoding a MeV H protein. The fragment shall encode a polypeptide which still has the activities as specified above. Accordingly, the polypeptide may comprise or consist of the domains of the polypeptide of the present invention conferring the said biological activities. A fragment as meant herein, preferably, comprises at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences.

The HA-coding polynucleotides of the present invention either essentially consist of the aforementioned nucleic acid sequences or comprise the aforementioned nucleic acid sequences. Thus, they may contain further nucleic acid sequences as well. Specifically, the HA-coding polynucleotides of the present invention may encode fusion proteins wherein one partner of the fusion protein is a polypeptide being encoded by a nucleic acid sequence recited above. Such fusion proteins may comprise as additional part polypeptides for monitoring expression (e.g., green, yellow, blue or red fluorescent proteins, alkaline phosphatase and the like) and/or so called “tags” which may serve as a detectable marker or as an auxiliary measure for purification purposes. Tags for the different purposes are well known in the art and comprise FLAG-tags, 6-histidine-tags, MYC-tags and the like. Preferably, the HA-coding polynucleotides comprise sequences encoding specificity domains, i.e. polypeptides that can mediate binding of the MeV to receptors different from the cognate receptor of MeV. More preferably, said specificity domain is a single-chain antibody, and most preferably it is a single-chain antibody which specifically recognizes the prostate stem cell antigen, PSCA (SEQ ID NO: 1; FIG. 1B, see also Reiter et al. (1998) PNAS 95, 1735-1740; Argani et al. (2001) Cane Res 61, 4320-4324; Sequence of the PSCA precursor: SEQ ID NO: 5, Genbank Acc no. AAH65183.1 GI:40850888).

It is to be understood that the current invention also relates to a measles viruses comprising said specificity domains as separate entities, i.e. not fused to the HA protein. Incorporation of said specificity domains into the virus separkely can be achieved by methods well known to the skilled artisan, e.g. by fusing the specificity domain to a different viral protein which will be incorporated into viral particles. Moreover, the viral hemagglutinin and/or the specificity domain can be encoded on the viral genome, such that progeny virus comprises the same proteins as the parental virus. However, the viral hemagglutinin and/or the specificity domain may also not be encoded on the viral genome, leading to the production of progeny virus being wildtype with respect to proteins whose modified version is not encoded on the viral genome.

The term “specifically recognizes” as used herein relates to an interaction between a molecule exposed on the surface of a cell and a specificity domain that leads to an at least fivefold, at least 10 fold, at least 25 fold, at least 50 fold, at least 100 fold increase of the binding of a MeV to a cell carrying said molecule as compared to a cell without said molecule.

The term “cell surface antigen specific for a solid cancer” as used in accordance with this specification relates to a macromolecule, preferably a protein, a glycoprotein, or a lipoprotein, exposed to the surface of a cell of a solid tumor. A “solid tumor”, preferably, is a neoplasia forming a solid mass at any place in the body of a patient. Preferably, said solid cancer is a prostate cancer, an ovarian cancer, or a lymphoma. More preferably, said solid tumor is a pancreas carcinoma. Most preferably, said tumor is a pancreas adenocarcinoma.

The definitions made above apply, mutatis mutandis, to the following embodiments:

In another preferred embodiment, the present invention relates to a polynucleotide coding for a recombinant MeV comprising a suicide gene in the viral genome, a viral hemagglutinin comprising at least one amino acid exchange preventing or reducing the binding activity of the said viral hemagglutinin, and/or a specificity domain which specifically recognizes a cell surface antigen specific for a solid tumor.

The nucleic acid sequence of measles virus is well known in the art (SEQ ID NO:6, Genbank Acc No: AF266290.1 GI:9181902 for strain Edmonston). The skilled artisan knows how to introduce the modifications to the polynucleotide sequence as described herein. An example can also be found in example 1A, SEQ ID NO:2.

The polynucleotides of the present invention shall be provided, preferably, either as an isolated polynucleotide (i.e. isolated from its natural context) or in genetically modified form. The polynucleotide, preferably, is DNA including cDNA, or RNA. The term encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides. In a preferred embodiment, the polynucleotide of the present invention further comprises measles virus nucleocapsid protein(s). In a more preferred embodiment, the measles virus nucleocapsid protein(s) are bound to the polynucleotide coding for said recombinant MeV, forming a MeV nucleocapsid.

In another preferred embodiment, the invention relates to a vector comprising the polynucleotide coding for a recombinant MeV.

The term “vector”, preferably, encompasses phage, plasmid, viral vectors as well as artificial chromosomes, such as bacterial or yeast artificial chromosomes. The vector encompassing the polynucleotides of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells.

More preferably, in the vector of the invention the polynucleotide is operatively linked to expression control sequences allowing expression in prokaryotic or eukaryotic cells or isolated fractions thereof. Expression of said polynucleotide comprises transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the T7, the lac, hp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise T7, tet, or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. Methods which are well known to those skilled in the art can be used to construct recombinant viral vectors. Preferably, the polynucleotide of the current invention is inserted in such a way into the vector that antigenomic RNAs are produced after induction of the aforementioned expression systems.

Another preferred embodiment of the current invention is a host cell comprising the recombinant measles virus of claim 1, the polynucleotide of claim 9, or the vector of claim 11.

As used in this specification, a “host cell” is a cell permissive for lytic replication of MeV. Preferably, the host cell is a mammalian or a monkey cell. More preferably, the host cell is a permissive tumor cell or a cultured cell from a cell line selected from the group consisting of B95a, Hela, Raji, Peer, Jurkat, and Molt-4. Most preferably, the host cell is a cultured cell from the Vero cell line. It is, however, also contemplated by the current invention that the host cell is a rescue cell line, expressing MeV proteins N, P, and L and allowing for transcription of antigenomic MeV RNAs, such as the 293-3-46 cell line (Radecke et al. (1995), EMBO J 14(23):5773-5784).

In another preferred embodiment, the present invention relates to a method for the manufacture of a recombinant measles virus, comprising the steps of a) culturing a host cell comprising the polynucleotide of claim 9 or 10 or the vector of claim 11 and b) obtaining the recombinant measles virus encoded by said polynucleotide or vector from the host cell.

As used herein, the ten “obtaining the recombinant measles virus” relates to the separation of MeV particles from the mixture used to produce said particles. Preferably, said separation preserves infectivity and replication competency of the MeV particles obtained. Methods to separate MeV from culture supernatant are well known in the art as described in Udem (1984) J Virol Methods 8, 123-136). It is to be understood that the degree of separation of MeV particles from the mixture used to produce said particles depends on the intended use of the MeV. Thus, separation may consist of only a centrifugation step removing cells from the supernatant. It may, however, also contain further separation steps like density gradient centrifugation.

In a further preferred embodiment, the current invention relates to a medicament comprising the recombinant measles virus of claim 1, the polynucleotide of claim 9 or 10, the vector of claim 11, the host cell of claim 12, or the recombinant measles virus produced by the method of claim 13.

The term “medicament” as used herein comprises the compounds of the present invention and optionally one or more pharmaceutically acceptable carrier. The compounds of the present invention can be formulated as pharmaceutically acceptable salts. Acceptable salts comprise acetate, methylester, HCl, sulfate, chloride and the like. Preferably, however, the compounds of the present invention are formulated as solutions. The medicaments are, preferably, administered topically or systemically. Suitable routes of administration conventionally used for drug administration are oral, intravenous, or parenteral administration as well as inhalation. However, depending on the nature and mode of action of a compound, the medicaments may be administered by other routes as well. For example, polynucleotide compounds may be administered in a gene therapy approach by using viral vectors or viruses or liposomes.

Moreover, the compounds can be administered in combination with other drugs either in a common medicament or as separated medicaments wherein said separated medicaments may be provided in form of a kit of parts.

The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The phatinaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the combination. Examples of such diluents are distilled water, physiological saline, Ringer's solutions, dextrose solution, and Hank's solution. In addition, the medicament or formulation may also include other carriers, adjuvants, or nontoxic, nontherapeutic, nonimmunogenic stabilizers and the like.

Specific medicaments are prepared in a manner well known in the pharmaceutical art and comprise at least one active compound referred to herein above in admixture or otherwise associated with a pharmaceutically acceptable carrier or diluent. For making those specific medicaments, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

In another preferred embodiment, the present invention relates to a method for treating a solid cancer comprising applying a therapeutically effective amount of the recombinant measles virus of claim 1, the polynucleotide of claim 9 or 10, the vector of claim 11, the host cell of claim 12, or the recombinant measles virus produced by the method of claim 13 to a patient.

The term “treating” as used in accordance with the current specification refers to ameliorating the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

The term “applying” as meant herein is bringing an agent into contact with a subject to be treated. Preferably, application is systemic, e.g. by application as an infusion. It is, however, also contemplated by the present invention that the application is topical, e.g. at the site of a tumor or within the tumor itself.

A therapeutically effective amount refers to an amount of the compounds to be used in a medicament of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 to 1000 μg for polynucleic acids and vectors, and in the range of 1000 to 10¹⁰ cell infectious units (ciu, 1 ciu being the amount of virus required to infect one target cell) for viral particles; however, doses below or above this exemplary range are envisioned, especially considering the aforementioned factors. Progress can be monitored by periodic assessment.

The medicaments and formulations referred to herein are administered at least once in order to treat or ameliorate or prevent a disease or condition recited in this specification. However, the said medicaments may be administered more than one time, for example from one to four times daily up to a non-limited number of days.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### FIGURE LEGENDS

FIG. 1: Schematic representation of the construction of recombinant measles virus

FIG. 2: Syncytia formation caused by retargeted MeV in various cell lines

FIG. 3 Graph showing cell viability measured quantitatively via XTT assay.

### EXAMPLES

### Example 1

### Antibody-Targeted Cell Fusion

In order to ablate the natural viral tropism, CD46 and SLAM binding sites were mutagenized. Additionally, a single-chain antibody (scFv) against PSCA was used to achieve a new tropism (SEQ ID NO: 1: amino acid sequence of the anti-PCSA-scFv-HA-Fusion; SEQ ID NO:2 nucleotide sequence of the anti-PCSA-scFv-HA-Fusion ORF). The eukaryotic expression plasmid pCG-HαPSCA was constructed encoding an H with particular mutations linked to the scFv against PSCA. In order to check biological function, it was co-transfected with a plasmid encoding the F protein, together with a EGFP expression plasmid (for monitoring transfection efficiency), into PSCA-positive and control cells. The cells were assessed for functional HαPSCA-triggered cell fusion by inspection of syncytia formation (Example 3). Only in the case of co-transfected cells presenting the PSCA (Capan-1, BxPC-3) or the pseudo receptor (Vero-αHis), syncytia formation could be observed, in contrast to PSCA-negative cells (Vero, 293).

### Example 2

### MeV Genome for Targeted Infection of PSCA-Presenting Cells

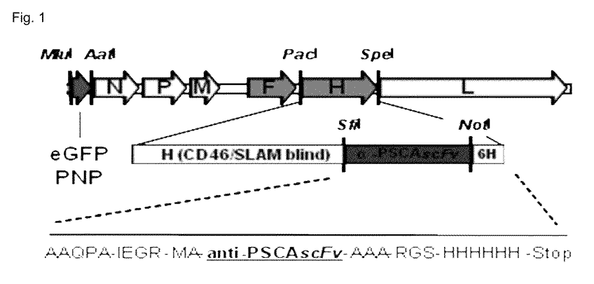

The eukaryotic expression vector pCG-HαPSCA—encoding the double-blind H fused to the scFv against PSCA—served as a donor to create recombinant genomic MeV cDNA constructs (FIG. 1). The PacI/SpeI-digested fragment from pCG-HαPSCA was exchanged against the corresponding fragment of p(+)MV-EGFP HαCD20, p(+)MV-PNP HαCD20 (Ungerechts et al. Cancer Res. 2007, 67:10939-10947), p(+)MV-bCD HαCEA, and p(+)MV-βG, respectively. The resulting full-length complementary DNAs were named p(+)MV-EGFP-HαPSCA, p(+)MV-PNP-HαPSCA, p(+)MV-bCD-HαPSCA, and p(+)MV-βG-HαPSCA (FIG. 1). Recombinant viruses were rescued as described previously (Radecke et al. EMBO J 1995, 14: 5773-5784) with slight modifications (Parks et al. J. Virol. 1999, 73:3560-3566). Propagation of initially generated, retargeted viruses was performed on the pseudo receptor-presenting Vero-αHis cells (Nakamura et al. Nat Biotech 2005, 23: 209-214). To prepare virus stocks, Vero-αHis cells were infected at an MOI of 0.03 and incubated at 37° C. for 36 hours. Viruses were harvested by one freeze-thaw cycle from their cellular substrate and resuspended in Opti-MEM. Titers were determined by 50% tissue culture infectious dose (TCID₅₀) titration on Vero-αHis cells.

### Example 3

### MeV Entry Through Targeted Receptor Addressing

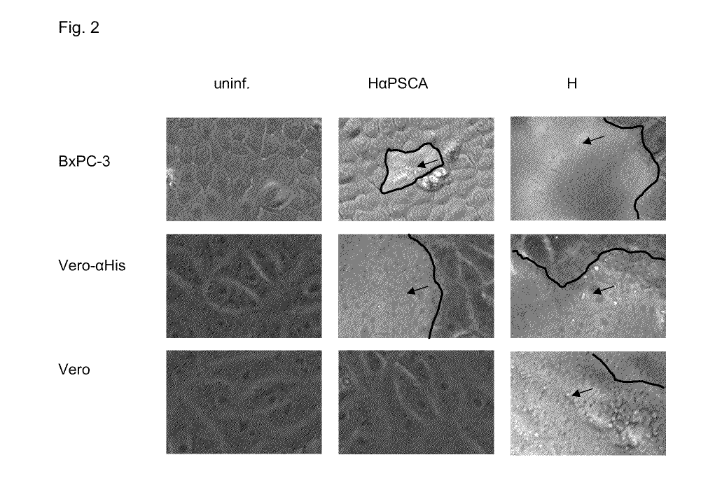

10⁵ cells each were seeded into a 24-well plate and infected with the retargeted MeV-EGFP HαPSCA or the MeV-EGFP harboring the unmodified H at a MOI=0,1. Syncytia formation was monitored and pictures were taken 48 h p.i. using a fluorescence microscope (FIG. 2). Representative areas with syncytia formation and green fluorescence are framed and indicated with black lines and arrows, respectively. The double-blind, retargeted HαPSCA virus could infect and induce syncytia formation in cells presenting the tumor antigen PSCA (BxPC-3) or the pseudo receptor (Vero-αHis), but was efficiently detargeted from parental Vero cells. In contrast, the unmodified MeV-EGFP was able to enter all cell types—via the normal route of infection using CD46—inducing a cytopathic effect.

TABLE 1
10⁴ cells each were seeded into a 96-well plate and infected
with the retargeted MeV-EGFP HαPSCA or the MeV-EGFP harboring
the unmodified H at a MOI = 0.1 (1000 cell infectious units).

| | Syncytia | | | Nuclei/syncytium | | |
|---|---|---|---|---|---|---|
| | BxPC-3 | Vero-His | Vero | BxPC-3 | Vero-His | Vero |
| HαPSCA | 220 | +++ | — | 4-16 | >> | — |
| H | +++ | +++ | +++ | 100 | >> | >> |

The total number of syncytia per 96-well was counted 48 h p.i. Samples where more than 90% of the cells were present as syncytia, thus not countable, are indicated as “+++”, whereas no syncytia formation observed is indicated as “—”. The average number of nuclei per syncytium is denoted and represented by “>>” in the case of massive syncytia formation with more than 100 nuclei and by “—” when no syncytia could be observed. The double-blind, retargeted HαPSCA virus could infect and induce syncytia formation in cells presenting the tumor antigen PSCA (BxPC-3) or the pseudo receptor (Vero-αHis), and was efficiently detargeted from parental Vero cells. In contrast, the unmodified MeV-EGFP was able to enter all cell types using the usual route of via CD46.

### Example 3

### Re-Targeted Infection and Suicide Gene/Prodrug Enhanced Oncolysis

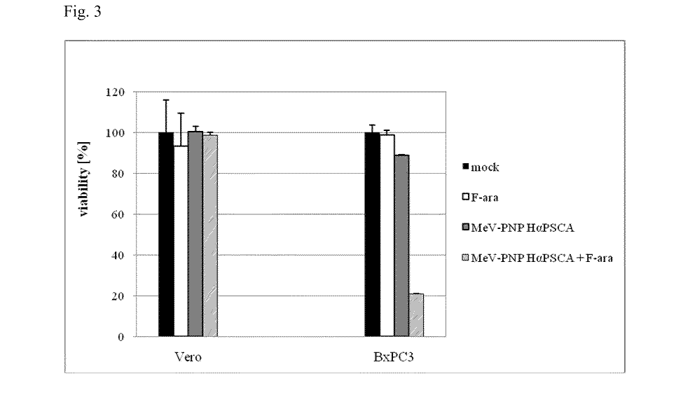

The pancreatic carcinoma cell line BxPC-3 was infected (MOI=1) with the PSCA-targeted MeV coding for the suicide gene purine nucleoside phosphorylase (PNP). For drug treatment, 5 μM Fludarabine (F-ara) was added and cell viability was measured quantitatively via XTT assay (72 h p.i.) (FIG. 3). The pancreatic carcinoma cell line BxPC-3 could be successfully infected with the double-blind, αPSCA-targeted virus and viral cytopathic effect could be enhanced up to nearly 5-fold after administration of the prodrug. Using the usually permissive host cell Vero for MeV as a control, neither infection nor toxic effects of converted prodrug could be observed showing the specifity of the αPSCA re-targeted virus.

1. A recombinant measles virus comprising
a) a suicide gene in the viral genome,
b) a viral hemagglutinin comprising at least one amino acid exchange preventing or reducing the binding activity of the said viral hemagglutinin, and
c) a specificity domain which specifically recognizes a cell surface antigen specific for a solid cancer.

2. The recombinant measles virus of claim 1, wherein the suicide gene is the E. coli purine nucleoside phosphorylase, cytosine deaminase, or the β-Glucuronidase.

3. The recombinant measles virus of claim 1, wherein the said at least one amino acid exchange abolishing the binding activity of the viral hemagglutinin is selected from the group consisting of R533A, Y481A, S548L, F549L, P497S and Y543A.

4. The recombinant measles virus of claim 1, wherein the single-chain antibody is fused to the viral hemagglutinin.

5. The recombinant measles virus of claim 1, wherein the single-chain antibody specifically recognizes the Prostate Stem Cell Antigen (PSCA).

6. The recombinant measles virus of claim 1, wherein the measles virus is derived from strain Edmonston-B.

7. The recombinant measles virus of claim 1, wherein the said hemagglutinin comprising at least one amino acid exchange and/or said single-chain antibody is encoded by the viral genome.

8. The recombinant measles virus of claim 1, wherein the said hemagglutinin comprising at least one amino acid exchange and/or said single-chain antibody is not encoded by the viral genome.

9. A polynucleotide coding for the recombinant measles virus of claim 1.

10. The polynucleotide of claim 9, wherein the polynucleotide further comprises measles virus nucleocapsid protein.

11. A vector comprising the polynucleotide of claim 9.

12. A host cell comprising the recombinant measles virus of claim 1.

13. A method for the manufacture of a recombinant measles virus, comprising the steps of
a) culturing a host cell comprising the polynucleotide of claim 9 or 10 or the vector of claim 11 and
b) obtaining the recombinant measles virus encoded by said polynucleotide or vector from the host cell.

14. A medicament comprising the recombinant measles virus of claim 1, the polynucleotide of claim 9 or 10, the vector of claim 11, or the host cell of claim 12.

15. A method for treating a solid cancer comprising applying a therapeutically effective amount of the recombinant measles virus of claim 1, the polynucleotide of claim 9 or 10, the vector of claim 11, or the host cell of claim 12.

16. The method of claim 15, wherein the cancer is pancreas carcinoma.


SEQUENCE LISTING (6 sequences)

SEQ ID NO: 1 - protein, 896 aa, Artificial Sequence
  Chemically synthesized anti-PCSA-scFv-HA-Fusion
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFTVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTNAAQPAIEGRGMAQVKLQESGGGLVQPGGSLKLSCVASGFTFSSYTMSWVRRTPEKRLEWVAYIHNGGGHTYYPDTIKGRFTISRDNAKNTLFLEMSSLKSEDTAMYYCTRRMYYGNSHWYFDVWGAGTSVTVSSAKTTPPSVYGGGGSGGGGSGGGGSTNSDIVMTQSPSSLSASLGDRVTINCRTSQDISNYLNWYQLTPDGTVKLLIYYTLKLNSGVPSRFSGSGSGTDYSLTINNLEKEDFATYFCQQSKTLPWTFGGGTKLEIKRAAARGSHHHHHH

SEQ ID NO: 2 - DNA, 2691 bp, Artificial Sequence
  Chemically synthesized anti-PCSA-scFv-HA-Fusion, coding sequence
atgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggaattcgacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaaccaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcactcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccgcggttgaacatgctgtggtttattacgtttacagcccaagccgcctatcgtcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatgcggcccagccggccatcgagggaaggggcatggctcaggtgaagctgcaggagtctgggggaggtttagtgcagcctggagggtccctgaaactctcctgtgtagcctctggattcactttcagttcctataccatgtcttgggttcgccggactccagagaagaggctggaatgggtcgcatacattcataatggtggtggtcacacctactatccagacaccataaagggccgattcaccatctccagagacaatgccaagaacaccctgttcctggaaatgagcagtctgaagtctgaagacacggccatgtattactgtacaagacgaatgtactacggtaatagccactggtacttcgatgtctggggcgcagggacctcggtcaccgtctcctcagccaaaacgacacccccatctgtctatggtggcggagggtctggtggcggagggtctggtggcggagggtcgacgaattctgatattgtgatgacacagtctccatcctccctgtctgcctctctgggcgacagagtcaccatcaattgcaggacaagccaggacattagcaactatttaaactggtatcagctgacaccagatggaactgttaaactcctgatctactacacattaaaattaaattcaggagtcccatcaaggttcagtggcagtgggtctgggacagattattctctcaccattaacaacctggagaaagaggattttgccacttatttttgccaacagagtaaaacacttccgtggacgttcggtggaggcaccaagctggaaatcaagcgggcggccgcaagaggttctcatcaccatcaccatcactaa

SEQ ID NO: 3 - DNA, 1854 bp, measles virus
atgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatag

SEQ ID NO: 4 - protein, 617 aa, measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTNRR

SEQ ID NO: 5 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 6 - DNA, 15894 bp, measles virus
  misc_feature: strain Edmonston
accaaacaaagttgggtaaggatagttcaatcaatgatcattttctagtgcacttaggattcaagatcctattatcagggacaagagcaggattaaggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtcaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgagaacaaggaaatctcagatattgaagtgcaagaccctgagggattcaacatgattctgggtaccatcctagctcaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcatcggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccctccatcattgttataaaaaacttaggaaccaggtccacacagccgccagcccatcaaccatccactcccacgattggagccgatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattatgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcctccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaagttccacaatgacagagatctacgacttcgacaagtcggcatgggacatcaaagggttgatcgctccgatacaacccaccacctacagtgatggcaggctggtgccccaggtcagagtcatagatcctggtctaggcgacaggaaggatgaatgctttatgtacatgtttctgctgggggttgttgaggacagcgatcccctagggcctccaatcgggcgagcatttgggtccctgcccttaggtgttggcaaatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcagtgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatagatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgatacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctccccttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagatgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaattaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagccagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaggccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatctcatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaattgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcctgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatacaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctaggttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaggagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctaacaaagatactatagagaagctagaggaattggcagccatcttatcgatggctctgctcctgggcaaaataggatcaatactggtgattaagcttatgcctttcagcggggattttgttcagggatttataagttatgtagggtcccattatagagaagtgaaccttgtataccctagatacagcaacttcatatctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattttgggggcacattcttctttactccgggaacagaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatagattaaagaaaactttgaaaatacgaagtttctattcccagctttgtctggt